(19) United States
(12) Patent Application Publication
(10) Pub. No.: US 2024/0252243 A1
(43) Pub. Date: Aug. 1, 2024

(54) Close-Packed Small Core Optical Fiber Bundles

(71) Applicant: C. R. Bard, Inc., Franklin Lakes, NJ (US)

(72) Inventors: Paul Thomas Mannion, Eliot, ME (US); Michal Tourchak, Atlanta, GA (US); Melody M. H. Kuroda, Durham, NC (US)

(21) Appl. No.: 18/566,030

(22) PCT Filed: May 31, 2022
(86) PCT No.: PCT/US22/31673

§ 371 Date: Nov. 30, 2023

Related U.S. Application Data
(60) Provisional application No. 63/195,329, filed on Jun. 1, 2021.

(51) Int. Cl. A61B 18/26; A61B 18/22

(52) U.S. Cl. CPC A61B 18/26; A61B 2018/2211; A61B 2018/263

(57) ABSTRACT

A medical instrument is disclosed that includes an elongate flexible shaft, a plurality of optical fibers extending along the length, and a laser control module coupled with the optical fibers. The instrument is configured for insertion into a patient body and/or into a working channel of an endoscope (e.g., ureteroscope). The instrument is configured for ablation of body tissue and/or a foreign substance within the body. The optical fibers can define a cross-sectional diameter within a range of 50 μm to 150 μm. Three or more of the optical fibers can be bundled together defining a circumscribed circle having a cross-sectional diameter less than 500 μm. Some optical fibers are peripherally disposed along the shaft and are configured to direct light radially outward. A lumen extending along the length of the shaft is coupled with fluid port coupled with the shaft.

### PRIORITY

This application claims the benefit of priority to U.S. Provisional Application No. 63/195,329, file Jun. 1, 2021, which is incorporated by reference in its entirety into this application.

### BACKGROUND

Laser lithotripsy typically comprises inserting and advancing a delivery optical fiber through the vasculature of a patient such that the delivery optical fiber approaches a calculus within the vasculature. Additionally, light is propagated along the delivery optical fiber and delivered to the calculus to break it into smaller pieces or dust. Traditionally, Holmium:YAG (Ho:YAG) lasers have been utilized in laser lithotripsy applications. However, the smallest delivery optical fibers (delivery fibers) generally available for use with Ho:YAG lasers have a core diameter on the order of 270 μm. The cladding diameter of these delivery fibers is on the order of about 400 μm. Thus, the sizing of the cladding diameter is restrictive for applications where a working channel is extremely small, as laser lithotripsy. Crystal lasers (e.g., Nd:YAG lasers) have similar drawbacks and size constraints of corresponding delivery fibers to those of Ho:YAG lasers.

As is known, a fiber laser is a particular type of laser where the active gain medium may be an optical fiber that is doped with a rare-earth element (“active fiber”). Additionally, a delivery fiber is optically coupled to the fiber laser and the light generated by the fiber laser is propagated along the delivery fiber. Delivery fibers utilized with fiber lasers often have smaller cladding diameters than those used with Ho:YAG lasers.

However, merely utilizing a delivery fiber connected to a fiber laser for processes such as laser lithotripsy may still result in retropulsion and/or damage to tissue surrounding a calculus undergoing ablation. Thus, what are needed are systems, devices and methods comprising a fiber laser system that reduce retropulsion, provide for use-case laser firing configurations, provide for system configurations that permit the use of irrigation or suction in combination with optical fibers for ablation as well as other advantages.

### SUMMARY

Embodiments herein disclose systems and methods that utilize a fiber laser system having a rare-earth element as a dopant, such as for example, but not limited or restricted to, thulium, erbium, ytterbium, neodymium, dysprosium, praseodymium, etc. Specific embodiments of the disclosure relate to a fiber laser configured to operate with an optical fiber having thulium as a dopant (thulium optical fiber). Some specific embodiments of the disclosure relate to packaging of a plurality of delivery fibers into particular configurations for advancement within a patient vasculature where the plurality of delivery fibers are connected to a fiber laser system. Additional embodiments disclose configurations of elongate shafts that include a plurality of delivery fibers configured for use with a fiber laser system. In some embodiments, the plurality of delivery fibers may be closely packed. In some embodiments, each of the optical fibers may have a 50 μm core diameter and a 74 μm cladding diameter. In other embodiments, the elongate shaft may include a plurality of optical fibers surrounding an irrigation and/or suction lumen.

Briefly summarized, disclosed herein is a medical instrument. The medical instrument includes an elongate flexible shaft defining a length extending between a proximal end and a distal end, a plurality of optical fibers extending along the length, and a laser control module including a laser light source operatively coupled with the optical fibers.

The instrument is configured for insertion into a patient body and/or into a working channel of an endoscope. The endoscope may be a ureteroscope. The instrument is configured for ablation of body tissue and/or a foreign substance within the body, such as a calculus.

The optical fiber may define a diameter within a range of 150 μm to 50 μm. One or more of the plurality of optical fibers may be located centrally along a longitudinal axis of the shaft. In some embodiments, three or more of the optical fibers may be disposed laterally adjacent one another to define a bundle of optical fibers and in some embodiments, the bundle may define a circumscribed circle having a diameter less than 1 mm. The optical fibers of the bundle may be configured to direct light distally away from the distal end of the shaft.

In some embodiments, three or more of the optical fibers are peripherally disposed along the shaft to define a peripheral set of the optical fibers. The optical fibers of the peripheral set may be configured to direct light radially outward from the shaft at the distal end of the shaft.

The instrument may further include a lumen extending along the length of the shaft and a fluid port coupled with the shaft, so that the fluid port is in fluid communication with the lumen. The lumen may be an annular lumen located radially outward of the bundle and the lumen may be located radially inward of the peripheral set. In some embodiments, the instrument may include a plurality of lumens located radially inward of the peripheral set.

In some embodiments, the instrument includes a hollow outer shaft and the shaft may be disposed within the outer shaft. In such an embodiment, the lumen is defined by an annular space between the shaft and the outer shaft, the fluid port is attached to the outer shaft, and the outer shaft is longitudinally displaceable with respect to the shaft.

Also disclosed herein is another embodiment of a medical instrument that includes an elongate flexible shaft defining a length extending between a proximal end and a distal end, a single optical fiber extending along the length, a fluid lumen extending along the length, and a laser control module including a laser light source operatively coupled with the optical fiber.

Also disclosed herein is a method of providing treatment to a urinary tract of a patient. The method includes advancing an elongate medical device along the urinary tract and positioning the distal end of the device at a desired location within the urinary tract. The device includes a plurality of optical fibers extending along an elongate shaft of the device to a distal end of the device and a laser control module disposed at a proximal end of the device, the control module including a corresponding plurality of light sources individually coupled with the plurality of optical fibers. The method further includes propagating laser light along one or more of the optical fibers to define an ablation within the urinary tract in accordance with the treatment.

In some embodiments of the method, one or more optical fibers define a first set of fibers that are configured to direct light distally away from the distal end. Similarly, one or more optical fibers define a second set of fibers that are configured to direct light radially away from the shaft at the distal end.

In some embodiments of the method, the device includes a lumen extending along the shaft between the proximal end and the distal end of the shaft and a fluid port coupled with the shaft, so that the fluid port is in fluid communication with the lumen.

The method may further include coupling a fluid device to the fluid port and passing a liquid through the lumen, wherein passing the liquid through the lumen cools the optical fibers.

In some embodiments of the method, the treatment includes a laser lithotripsy of a calculus disposed within the urinary tract, and propagating laser light along the one or more optical fibers includes propagating laser light along the optical fibers of the first set to impinge light onto the calculus to form a hole in the calculus. In such an embodiment, positioning the distal end of the device at a desired location includes inserting the distal end of the device within the hole of the calculus, and propagating laser light along the one or more optical fibers includes propagating laser light along the optical fibers of the second set to impinge light onto an inside surface of the hole in the calculus to break the calculus apart into pieces.

The method may further include creating a suction within the lumen to draw the calculus toward the distal end of the shaft and/or to transport calculus pieces proximally along the lumen.

In some embodiments, positioning the distal end of the device at a desired location includes positioning the distal end within the prostate, and propagating laser light along the one or more optical fibers includes propagating laser light along the optical fibers of the second set to impinge light onto an inside surface of the prostate to ablate prostate tissue in accordance with the treatment.

In some embodiments of the method, the device includes a hollow outer shaft. In such an embodiment, the shaft is disposed within the outer shaft so that the lumen is defined by an annular space between the shaft and the outer shaft. The fluid port is coupled with the outer shaft, and the outer shaft is longitudinally displaceable with respect to the shaft. In such an embodiment, the method further includes displacing the outer shaft with respect to the shaft.

These and other features of the concepts provided herein will become more apparent to those of skill in the art in view of the accompanying drawings and following description, which disclose particular embodiments of such concepts in greater detail.

### BRIEF DESCRIPTION OF THE DRAWINGS

Embodiments of the disclosure are illustrated by way of example and not by way of limitation in the figures of the accompanying drawings, in which like references indicate similar elements and in which:

FIG. 1A illustrates an embodiment of a medical instrument including optical fibers extending along an elongate shaft, in accordance with some embodiments;

FIGS. 1B-1E illustrate embodiments of a distal end view of the shaft of FIG. 1A, in accordance with some embodiments;

FIG. 2 is a distal end view of second embodiment of a shaft, in accordance with some embodiments;

FIG. 3 is a distal end view of third embodiment of a shaft, in accordance with some embodiments;

FIG. 4A is side view of fourth embodiment of a shaft, in accordance with some embodiments;

FIG. 4B is detail perspective view of a distal portion of the shaft of FIG. 4A, in accordance with some embodiments;

FIG. 4C is a distal end view of the shaft of FIG. 4A, in accordance with some embodiments; and

FIGS. 5A and 5B illustrate of an exemplary use case of the laser of FIG. 1A.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

The directional terms “proximal” and “distal” are used herein to refer to opposite locations on a medical device. The proximal end of the device is defined as the end of the device closest to the end-user when the device is in use by the end-user. The distal end is the end opposite the proximal end, along the longitudinal direction of the device, or the end furthest from the end-user.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method

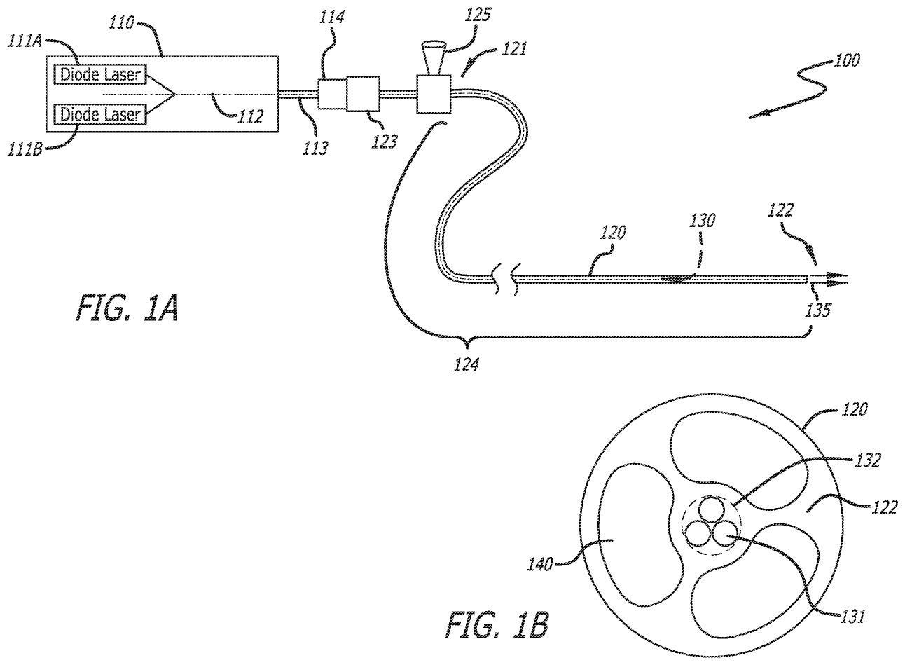

FIG. 1A illustrates an embodiment of a medical instrument 100, which may be a fiber laser system that includes a laser control module 110 that is coupled to an elongate shaft (shaft) 120, where the shaft 120 has disposed therein one or more optical fibers 130 (which may also be referred to as “delivery fibers”). In some embodiments, as illustrated in FIG. 1A, a first end of an optical interconnect 113 may be connected to the laser control module 100 and a second end of the optical interconnect 113 may be connected to a module connector 114, which may be connected to a shaft connector 123. The shaft 120 may extend a length 124 from the shaft connector 123. In some embodiments, the shaft 120 may include one or more lumens (see FIG. 1B) such that a fluid port 124 may be in fluid communication with the one or more lumens.

In some particular embodiments, the laser control module 110 may be a fiber laser that includes one or more diode lasers 111A-111B that may be electronically modulated. It should be understood that additional diode lasers may be coupled, e.g., 111A-111i (where i≥1). The diode lasers 111A-111B may be optically coupled with a rare-earth element doped silica fiber 112 may be utilized as a gain medium in order to generate a laser beam, which in the case of a fiber laser is typically a uniform laser beam (where the fiber 112 may be referred to as an “active fiber 112”). The uniform laser beam may be output from the laser control module 110 to the shaft 120, where in some embodiments the interconnects illustrated in FIG. 1A are optionally disposed between the laser control module 110 and the shaft 120. Each of the optical fibers 130 extend along at least a portion of the elongated shaft 120.

In some embodiments, the instrument 100 may be employed to perform medical procedures associated with a urinary tract of a patient body. The procedures may include laser lithotripsy, treating benign prostatic hyperplasia, or other medical procedures that include the ablation of body tissue and/or foreign substances. In some instances, the medical instrument 100 may be used in conjunction with an endoscope (e.g., a ureteroscope) during the performance of the medical procedure. For example, in some instances the medical instrument 100 may be inserted through a working channel of the endoscope.

In some embodiments, as noted above, the elongate flexible shaft 120 is operatively coupled with a laser control module 110 via a module connector 114 (also referred to herein as a “fiber optic connector”) connected to a shaft connector 123. In some embodiments, an optical interconnect 113 may be disposed between the laser control module 110 and the module connector 114. The interconnect 113 may be flexible and relatively long (e.g., about two to ten feet in length) so that the laser control module 110 may be located away from the patient for convenience. The laser control module 110 includes one or more diode lasers 111A-111B configured to pump light into and stimulate radiation emission within active fiber 112 generating a laser beam, which then propagates distally along the delivery fibers disposed within the shaft 120. The interconnect 113 includes one or more optical fibers for propagating the light from the diode lasers 111A-111B to the optical fibers 130. The laser controller 110 may include multiple light sources (e.g., diode lasers 111A-111i). In some embodiments, a first diode laser 111A may correspond to a first optical fiber 130 and a second diode laser 111B may correspond to a second optical fiber 130. In some embodiments, the laser controller 110 may be configured to activate the diode lasers 111A-111B individually or as groups. In some embodiments, the laser controller 110 may activate the diode lasers 111A-111B at a pulse repetition rate of up to 2000 Hz or more with an energy per pulse as low as 0.025 Joules. In some embodiments, the laser controller 110 may activate the diode lasers 111A-111B to propagate laser light to individual or a subset of the optical fibers 130 in a selective manner. Examples of configurations of the plurality of optical fibers 130 are discussed below with respect to, for example, FIGS. 1B-4B.

The fibers 130 may be end-firing (or end on firing) fibers. In other words, the fibers 130 may be configured to direct light 135 distally away from the distal end 122 of the shaft 120.

The shaft 120 is configured for insertion into a urinary tract of the patient body. As such, the shaft 120 defines a length 124 extending between a proximal end 121 and a distal end 122, and the length 124 is sufficient to extend from a location outside the patient to a location within a kidney of the patient. As stated above, the shaft 120 may be inserted into a working channel of a ureteroscope. As such, the length 124 may exceed the length of the ureteroscope, and a cross-sectional diameter of the shaft 120 may be sized for insertion into the working channel, i.e., less than a diameter of the working channel. In some embodiments, the cross-sectional diameter of the shaft 120 may be substantially less than the diameter of the ureteroscope. The diameter of the shaft 120 may be less than about 1.2 mm, 600 μm, 300 μm, or 150 μm. The relatively small diameter of the shaft 120 with respect to the inside diameter of the ureteroscope may provide for enhanced fluid flow through the working channel with the shaft 120 disposed therein.

FIG. 1B illustrates a first embodiment of a distal end view of the shaft 120, in accordance with some embodiments. The one or more optical fibers (fibers) 130 extend along the length 124 of the shaft 120 to the distal end 122. The shaft 120 may include 1, 2, 3, 4, 5, or more fibers 130. In some embodiments, the shaft 120 may include up to 10, 20, 30 or more fibers 130. The fibers 130 may have a cross-sectional diameter of less than about 150 μm, 100 μm 75 μm, or 50 μm (e.g., may have a cross-sectional diameter within a range of 50 μm to 150 μm).

In some embodiments, two or more fibers 130 may be disposed laterally adjacent each other to form a close-packed bundle of fibers 130. For example, as shown in FIG. 1B, three or more fibers 130 may form a bundle 131. The bundle 131 may be centrally disposed within the cross section of the shaft 120 or at any other location across the cross section. Other fibers 130 may be disposed at other locations across the cross section either individually or in bundles. In some embodiments, a circle 132 circumscribing the bundle 131 may be less than 1 mm, 500 μm, 250 μm, 225 μm, 200 μm, 180 μm, or 160 μm.

The shaft 120 may include one or more lumens 140 extending along a length thereof between a fluid port 125 (FIG. 1A) and the distal end 122. The port 125 is in fluid communication with the lumens 140. The lumens 140 may be disposed radially outward with respect to the bundle 131. As shown in FIG. 1B, the shaft 120 may include 3 lumens. In other embodiments, the shaft 120 may include one, two, three, four, five or more lumens 140. The lumens 140 may be configured to provide cooling to the fibers 130. During operation, as the stimulated emission of radiation takes place within each fiber 130, heat may be generated causing the temperature of the fiber 130 to exceed a desired working temperature. As such, the lumens 140 (or the shaft 120 generally) may be configured so that in use, liquid passing through the lumens 140 may induce thermal energy transfer away from the fibers 130 thereby cooling the fibers 130. FIG. 1C illustrates a second embodiment of a distal end view of the shaft 120, in accordance with some embodiments. In some exemplary embodiments, each of the fibers illustrated in the embodiments of FIGS. 1B-1C may be side-firing fibers.

FIG. 1D illustrates a third embodiment of a distal end view of the shaft 120, in accordance with some embodiments. The embodiment of FIG. 1D provides a physician or other medical professional the ability to apply laser energy in a particular using a subset of the fibers. For example, laser energy may be applied by activation of a first subset of the bundle 131 through fibers 130A-130D while fibers 130E-130G are not activated. Similarly, laser energy may be applied by activation of a second subset of the bundle 131 through fibers 130A and 130E-130G while fibers 130B-130D are not activated. However, any combination of fibers 130A-130G may be activated to apply laser energy. Stated differently, a first subset may be activated to apply laser energy while a second subset is not activated. Such an embodiment advantageously enables the physician or other medical professional to apply laser energy while protecting surrounding tissue.

FIG. 1E illustrates a fourth embodiment of a distal end view of the shaft 120, in accordance with some embodiments. Such an embodiment may be utilized for treating larger kidney stones where, for example, a first fiber 130A may be an end-on firing fiber configured to “bore” a hole in the kidney stone (not shown) such that the bundle of fibers 131 may be positioned within the bore. Additionally, the second-fifth fibers 130B-130E may be side-firing fibers configured to ablate the kidney stone from the bore therein. Such an embodiment may advantageously reduce retropulsion as the ablation forces would be equalized on the kidney stone. The embodiment of FIG. 1E may be used in other situations such as, for example, with treatment of benign prostatic hyperplasia (BPH).

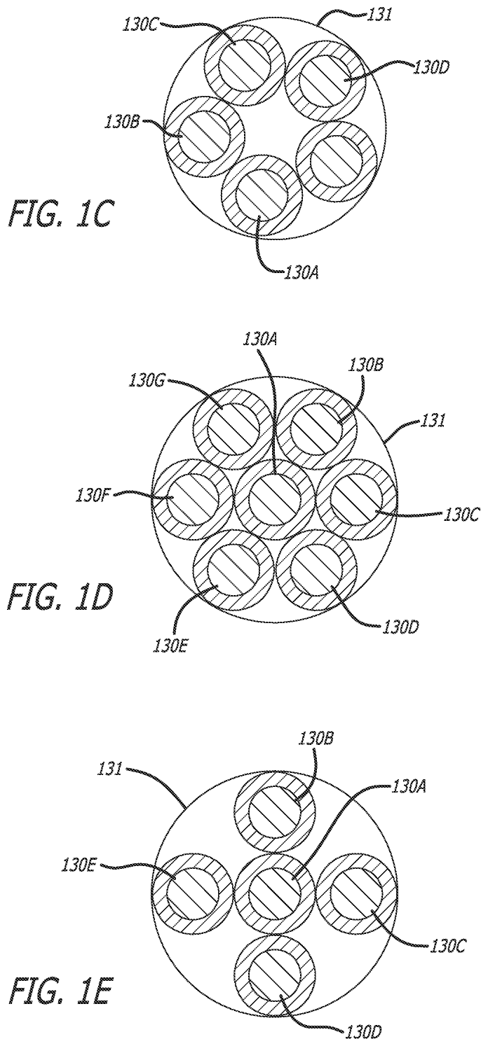

FIG. 2 illustrates another embodiment of a shaft 220 that may be include by the system 100. The shaft 220 can, in certain respects, resemble components of the shaft 120 described in connection with FIGS. 1A-1B. It will be appreciated that all the illustrated embodiments may have analogous features. Accordingly, like features are designated with like reference numerals, with the leading digits increment to “2.” For instance, the lumen is designated as “140” in FIGS. 1A-1B, and an analogous lumen is designated as “240” in FIG. 2. Relevant disclosure set forth above regarding similarly identified features thus may not be repeated hereafter. Moreover, specific features of shaft 120 and related components shown in FIGS. 1A-1B may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the shaft 220. Any suitable combination of the features, and variations of the same, described with respect to the shaft 120 combination and components illustrated in FIGS. 1A-1B can be employed with the shaft 220 and components of FIG. 2, and vice versa.

FIG. 2 is an end view illustration of the shaft 220. The shaft 220 includes a centrally located lumen 240 and one or more fibers 230 (designated in sets as 230A, 230B) disposed radially outward of the lumen 240. The shaft 220 may include 1, 2, 3, 4, 5, or more fibers 230. In some embodiments, the shaft 220 may include up to 10, 20, 30 or more fibers 230. In some embodiments, the fibers 230 may be combined into one or more close-packed fiber bundles (not shown).

In some embodiments, the fibers 230 may be divided into subsets. For example, a first subset 230A of the fibers 230 may be end-firing fibers. In other words, the first subset of fibers 230A may be configured to direct light 235 distally away from the distal end 222 of the shaft 220. (i.e., out of the page). A second subset 230B of fibers 230 may be configured to direct light 235 radially/laterally away from the shaft 220. In use, the laser control module 110 may individually activate the fibers 230 of subsets 230A, 230B at different times. For example, the laser control module 110 may activate the first subset 230A of fibers 230 while maintaining deactivation of the second subset 230B of fibers 230 and vice versa. In other embodiments, the laser control module 110 may activate all fibers 230 at the same time. The shaft 220 may further include other fibers 230 not include the subsets 230A, 230B.

The shaft 220 may be configured to allow light 235 to laterally pass through the shaft material from the fiber 230 to an external surface 226 of the shaft 220. In some embodiments, the shaft 220 may include openings (not shown) to provide a pathway for the light 235. In other embodiments, the shaft 220 or portions thereof may be formed of any material suitably transparent to the light 235 such as acrylic or polycarbonate, for example.

The lumen 240 extends the length of the shaft 220 between a fluid port (not shown, but see FIG. 1A) and the distal end 222. The lumen 240 may be configured to provide cooling to the fibers 230. During operation, as the stimulated emission of radiation takes place within each fiber 230, heat may be generated causing the temperature of the fiber 230 to exceed a desired working temperature. As such, the lumen 240 (or the shaft 120 generally) may be configured so that in use, liquid passing through the lumen 240 may induce thermal energy transfer away from the fibers 230 thereby cooling the fibers 230. The lumen 240 may also provide a pathway for ablated substances such as calculus dust to be transported proximally along the shaft 220 and out of the body.

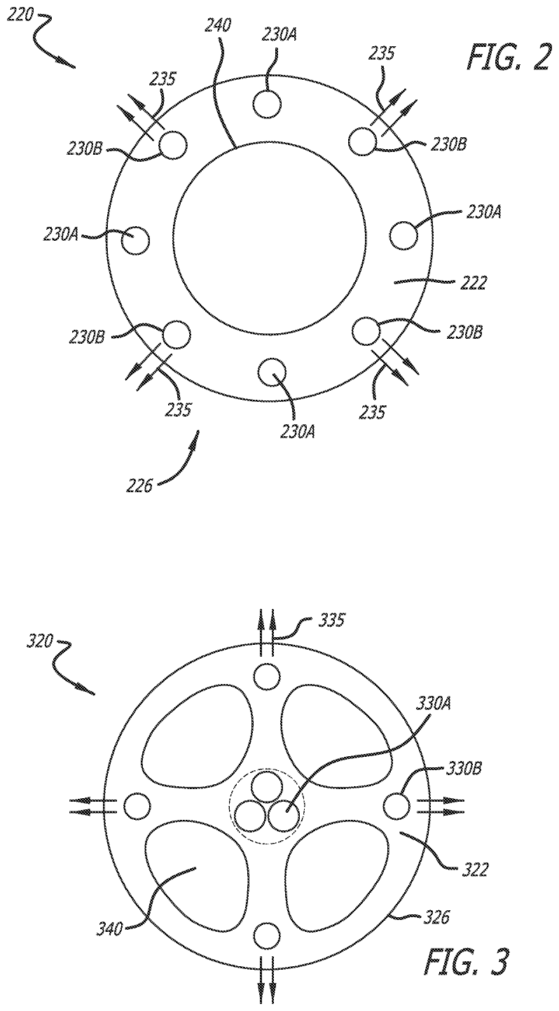

FIG. 3 is an end view illustration of another embodiment of a shaft 320 that may be included by the system 100. The fibers 330 of the shaft 320 may be divided into subsets. For example, a first subset of fibers 330A may be end-firing fibers centrally located within the shaft 320. A second subset of fibers 330B may be side-firing fibers disposed adjacent an outside surface 326 of the shaft 320. Any subset of the fibers 330 may be combined into close-packed fiber bundles.

In use, the laser control module 110 (FIG. 1A) may activate the fibers 330A, 330B at different times. For example, the laser control module 110 may activate the end-firing fibers 330A while maintaining deactivation of the side-firing fibers 330B and vice versa. In other embodiments, the laser control module 110 may activate the fibers 330A, 330B at the same time. Any of the fibers 330 may be activated individually or in other groupings.

The shaft 320 may be configured to allow light 335 to laterally pass through the shaft material from the side-firing fibers 330B to an external surface 326 of the shaft. In some embodiments, the shaft 320 may include openings (not shown) to provide a pathway for the light 335. In other embodiments, the shaft 320 may be formed of a material suitably transparent to the light 335.

The shaft 320 further includes one or more lumens 340 extending the length of the shaft 320 between a fluid port (not shown, but see FIG. 1A) and the distal end 322. The lumens 340 may be interspersed between the fibers 330A, 330B.

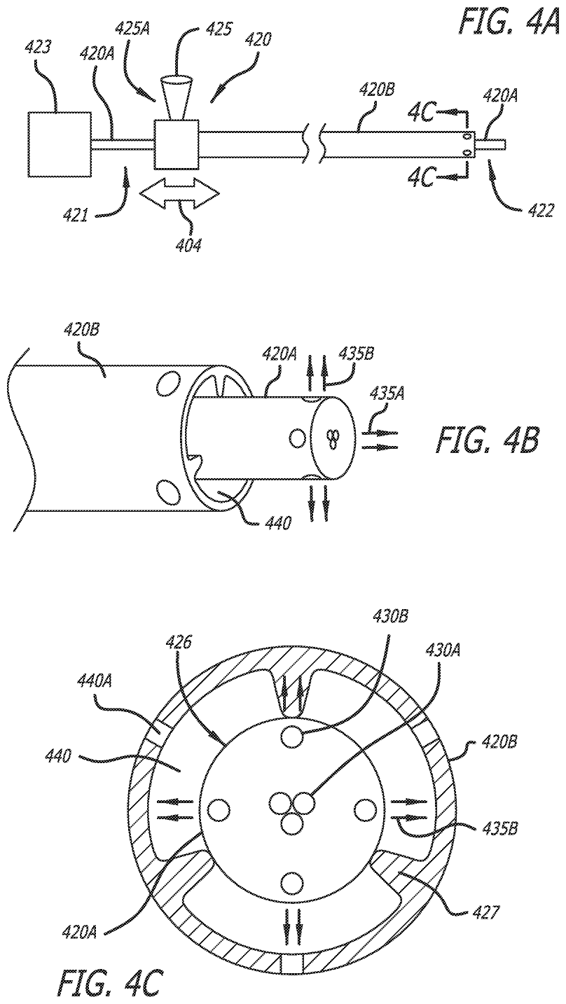

FIGS. 4A-4C illustrate another embodiment of a shaft 420 that may be included by the system 100. FIG. 4A is a side view of the shaft 420, FIG. 4B is a detail side perspective view of a distal portion of the shaft 420, and FIG. 4C is distal end view of the shaft 420 with the outer shaft 420B shown in cross-section cut along sectioning lines 4C-4C of FIG. 4A. The shaft 420 includes an inner shaft 420A and an outer shaft 420B. The outer shaft 420B is slidably coupled with the inner shaft 420A so that the outer shaft 420B may be displaced longitudinally along the inner shaft 420A as indicated by the arrow 404. In use, the outer shaft 420B may be displaced distally along the inner shaft 420A so that a distal end of the outer shaft 420B extends beyond the inner shaft 420A. Alternatively, the outer shaft 420B may be displaced proximally along the inner shaft 420A so that a distal end of the inner shaft 420A extends beyond the outer shaft 420B. A shaft coupling 423 is shown disposed at the proximal end 421 of the inner shaft 420A.

The shaft 420 is configured to define a lumen 440 between the outer shaft 420B and the inner shaft 420A. The outer shaft 420B includes a fluid port 425 disposed at a proximal end of the outer shaft 420B and the fluid port 425 is in fluid communication with the lumen 440. The fluid port 425 also includes a sliding fluid seal 425A between the outer shaft 420B and the inner shaft 420A to define a proximal end of the lumen 440. The outer shaft 420B may include protrusions 427 extending inward to the inner shaft 420A to concentrically constrain the inner shaft 420A with respect to the outer shaft 420B. In alternative embodiments, the protrusions 427 may extend outward from the inner shaft 420A to the outer shaft 420B. The outer shaft 420B may include one or more openings 440A extending through an annular wall of the outer shaft 420B to define radially oriented fluid pathways extending between the lumen 440 and an exterior of the outer shaft 420B. In use, a clinician may couple a fluid device (e.g., a syringe) with the fluid port 425 and push liquid distally through the lumen 440 so that the liquid exits through the openings 440A and/or the end of the outer shaft 420B. The clinician may also draw liquid proximally through the lumen 440.

The inner shaft 420A includes a plurality of fibers 430 which may be divided into one or more end-firing fibers 430A centrally located within the inner shaft 420A and one or more side-firing fibers 430B disposed adjacent an outside surface 426 of the inner shaft 420B. Any subset of the fibers 430 may be combined into close-packed fiber bundles.

In use, the laser control module 110 may activate the fibers 430A, 430B at different times. For example, the laser control module 110 may activate the end-firing fibers 430A to direct light 435A distally away from the inner shaft 420A while maintaining deactivation of the side-firing fibers 430B and vice versa. In other embodiments, the laser control module 110 may activate the fibers 430A, 430B at the same time. Similarly, the laser control module 110 may activate a subset of end-firing fibers 430A or the side-firing fibers 430B while maintaining deactivation of another subset of the end-firing fibers 430A or the side-firing fibers 430B. In other words, the laser control module 110 may activate any of the fibers 430 individually or in groupings.

The inner shaft 420A may be configured to allow light 435B to laterally pass through the shaft material from the side-firing fibers 430B to the exterior of the inner shaft 420B. In some embodiments, the inner shaft 420B may include openings (not shown) to provide pathways for the light 435B. In other embodiments, the shaft 420B may be formed of a material suitably transparent to the light 435B.

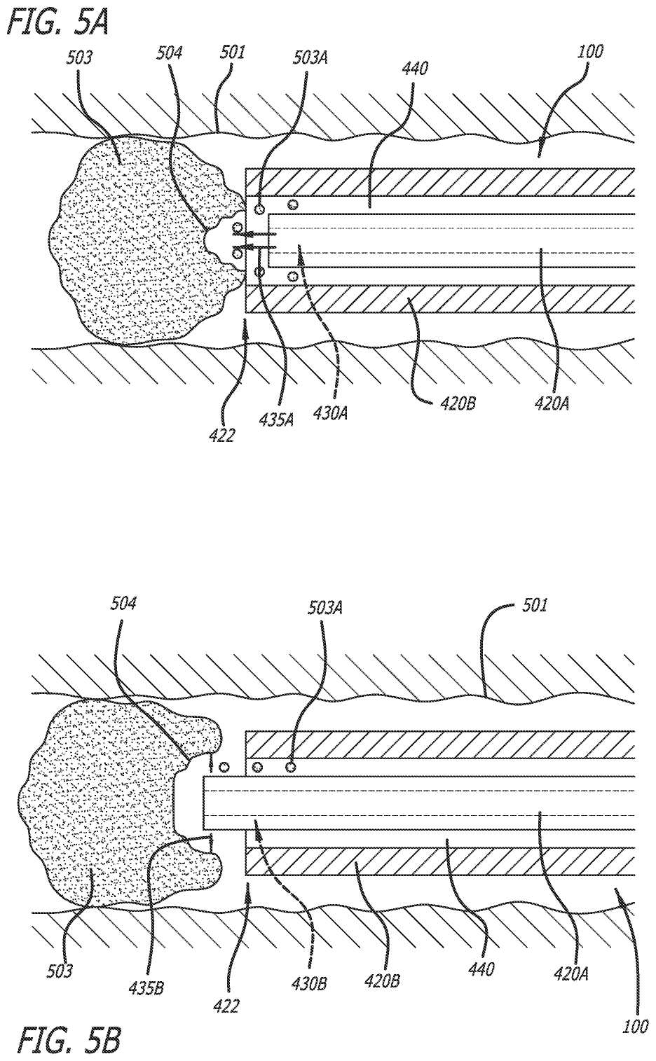

FIGS. 5A and 5B illustrate an exemplary use case of the system 100 including the shaft 420. The use case employs the system 100 to perform a laser lithotripsy of a calculus 503. As shown in FIG. 5A, the shaft 420 is inserted into a urinary tract 501 so that the distal end 422 is positioned adjacent the calculus 503. In some instances, the outer shaft 420B may be distally displaced so that the distal end of the outer shaft 420B extends beyond the inner shaft 420A. In addition to performance of laser lithotripsy of a calculus, the system 100 may be utilized to perform such a procedure on various mineral deposits formed within a patient body. For example, the system 100 may be utilized to perform laser lithotripsy on mineral and salt deposits formed in a patient kidney, typically referred to as kidney stones.

The laser control module 110 (FIG. 1A) may activate end-firing fibers 430A to bore a hole 504 in the calculus 503. In some instances, the clinician may define a suction within the lumen 440 which may transport calculus pieces or dust 503A proximally through the lumen 440 and out of the patient. In some instances, the suction may draw the calculus 503 toward the distal end 422 of the shaft preventing repulsion of the calculus 503 during the boring process.

After the hole 504 is bored, the outer shaft 420B may be proximally displaced so that the distal end of the inner shaft 420A extends beyond the outer shaft 420B as shown in FIG. 5B. The distal end of the inner shaft 420A is disposed within the hole 504. With the distal end of the inner shaft 420A disposed within the hole 504, the side-firing fibers 430B may be activated to the break apart the calculus 503 and/or ablate the calculus 503 from the inside out. The side-firing fibers 430B may produce a force directed radially outward from the inner shaft 420A onto the calculus. As such, repulsion of the calculus 503 may prevented or minimized during lithotripsy. In some instances, the clinician may also define a suction within the lumen 440 to transport calculus dust proximally through the lumen 440 and out of the patient during the inside-out ablation process.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A medical instrument, comprising:
an elongate flexible shaft defining a length extending between a proximal end and a distal end;
a plurality of optical fibers extending along the length, wherein one or more of the plurality of optical fibers is has a cross-sectional diameter within a range of 150 μm to 50 μm;
a fiber optic connector disposed at the proximal end; and
a laser control module comprising a laser light source operatively coupled with the plurality of optical fibers.

2. The instrument of claim 1, wherein the instrument is configured for insertion into a patient body.

3. The instrument of claim 1, wherein the instrument is configured for insertion into a working channel of an endoscope.

4. The instrument of claim 3, wherein the endoscope is a ureteroscope.

5. The instrument of claim 1, wherein the instrument is configured for ablation of body tissue.

6. The instrument of claim 1, wherein the instrument is configured for ablation of a calculus.

7. The instrument of claim 1, wherein one or more of the plurality of optical fibers are located centrally along a longitudinal axis of the shaft.

8. The instrument of claim 1, wherein three or more of the plurality of optical fibers are disposed laterally adjacent one another to define a bundle of the plurality of optical fibers.

9. The instrument of claim 8, wherein the bundle defines a circumscribed circle having a diameter less than 1 millimeter.

10. The instrument of claim 8, wherein the optical fibers of the bundle are configured to direct light distally away from the distal end of the shaft.

11. The instrument of claim 8, wherein three or more of the optical fibers are peripherally disposed along the shaft to define a peripheral set of the optical fibers.

12. The instrument of claim 11, wherein the optical fibers of the peripheral set are configured to direct light radially outward from the shaft at the distal end of the shaft.

13. The instrument of claim 8, further comprising a lumen extending along the length.

14. The instrument of claim 13, further comprising a fluid port coupled with the shaft, the fluid port in fluid communication with the lumen.

15. The instrument of claim 13, wherein the lumen is an annular lumen located radially outward of the bundle.

16. The instrument of claim 13, wherein the lumen is located radially inward of the peripheral set.

17. The instrument of claim 14, further comprising a plurality of lumens located radially inward of the peripheral set, the fluid port in fluid communication with the plurality of lumens.

18. The instrument of claim 14, further comprising a hollow outer shaft, wherein:
the shaft is disposed within the outer shaft,
the lumen is defined by annular space between the shaft and the outer shaft,
the fluid port is attached to the outer shaft, and
the outer shaft is longitudinally displaceable with respect to the shaft.

19. The instrument of claim 8, wherein the bundle includes a first subset of the plurality of optical fibers configured for activation at a first time and a second subset of the plurality of optical fibers configured for activation at a second time.

20. The instrument of claim 8, wherein the bundle includes an end-firing optical fiber and a plurality of side-firing fiber.

21. The instrument of claim 1, wherein the laser control module is a component of a fiber laser system and includes at least a first laser diode.

22-57. (canceled)
